(19) United States
(12) Patent Application Publication
(10) Pub. No.: US 2009/0270400 A1
(43) Pub. Date: Oct. 29, 2009

(54) PAINKILLING ASSOCIATION COMPRISING A DIHYDROIMIDAZOPYRAZINE DERIVATIVE

(75) Inventors: Michel Auguet, Palaiseau (FR); Christine Favre, Saint Maurice Montcouronne (FR); Gregoire Prevost, Antony (FR); Pierre-Etienne Chabrier De Lassauniere, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris (FR)

(21) Appl. No.: 11/722,070

(22) PCT Filed: Dec. 16, 2005
(86) PCT No.: PCT/FR2005/003162

§ 371 Date: Jun. 18, 2007

(30) Foreign Application Priority Data
Dec. 17, 2004 (FR) 0413453

(51) Int. Cl. A61K 31/495

(52) U.S. Cl. 514/249

(57) ABSTRACT

The invention relates to a product comprising (1R)-1-[(({2R)-2-amino-3-[(8S)-8-(cyclohexylmethyl) -2-phenyl-5,6-dihydroimidazo [1,2-a]pyrazin-7(8H)-yl]-3-oxopropyl}dithio)methyl]-2-[(8S)-8-(cyclohexylmethyl) -2-phenyl-5,6-dihydrolmidazo[1,2-a]pyrazin-7(8H)-yl]-2-oxoethylamine in association with an analgesic agent selected from morphine, the similar or a morphine derivative, sodium channel inhibitors, non-steroidal antiflammatory agents (AINS), glutamatergic system inhibitors, tricycle antidepressants and gabaergic derivatives for simultaneous therapeutic use which is separated or out over the time for pain treatment or prevention.

The present invention relates to painkilling combinations comprising a dihydroimidazopyrazine derivative, namely (1R)-1-[({(2R)-2-amino-3-[(8S)-8-(cyclohexylmethyl) -2-phenyl-5,6-dihydroimidazo[1,2-α]pyrazin-7(8H)-yl]-3-oxopropyl}dithio) methyl]-2-[(8S)-8-(cyclohexylmethyl)-2-phenyl-5,6-dihydroimidazo[1,2-α]pyrazin-7(8H)-yl]-2-oxoethylamine or one of its pharmaceutically acceptable salts.

Today, pain still remains a pathology which is difficult to relieve or cure. Use of currently available compounds which make it possible to reduce pain satisfactorily is often associated with undesirable side effects (sedation, habituation, hyperalgesia, risk of ulcers) In order to reduce these risks of side effects, several painkiller agents with different action mechanisms are often used in combination. This enables improved pain treatment, while reducing the risks of undesirable side effects, by using reduced doses of each agent.

(1R)-1-[({(2R)-2-amino-3-[(8S)-8-(cyclohexylmethyl)-2-phenyl -5,6-dihydroimidazo[1,2-α]pyrazin-7(8H)-yl]-3-oxopropyl}dithio)methyl]-2-[(8S) -8-(cyclohexylmethyl)-2-phenyl-5,6-dihydroimidazo[1,2-α]pyrazin-7(8H)-yl]-2-oxoethylamine has been described by the Applicant as an anti-cancer agent.

Morphine itself, which is well known today for its painkilling effects, was isolated from the opium of which it is the principal constituent very early in the 19th century by a German pharmacist, Friedrich Sertürner.

The Applicant has now discovered that the combination of (1R)-1-[({(2R)-2-amino-3-[(8S)-8-(cyclohexylmethyl)-2-phenyl-5,6-dihydroimidazo[1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine and morphine or a morphine analogue or derivative has a powerful synergic effect in the treatment of pain to the extent of reducing considerably the doses of morphine or morphine analogue or derivative administered to the patient, while retaining an equivalent analgesic effect. In fact, these two active ingredients administered in combination at sub-active doses (i.e. at doses which do not by themselves produce a therapeutic effect), produce a highly significant therapeutic effect when they are combined.

The present invention relates to a product comprising (1R)-1-[({(2R)-2-amino-3-[(8S)-8-(cyclohexylmethyl)-2-phenyl-5,6-dihydroimidazo[1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or one of its pharmaceutically acceptable salts in combination with an analgesic agent chosen from the ligands of opiate receptors or their salts, such as for example morphine or morphine derivatives, sodium channel inhibitors, non-steroidal anti-inflammatories (NSAID), inhibitors of the glutamatergic system, tricyclic antidepressants, alpha 2 adrenergic agonists, cannabinoids and GABAergic derivatives for therapeutic use which is simultaneous, separate or spread out over time for the treatment or prevention of pain.

By pharmaceutically acceptable salt is meant in particular addition salts with inorganic acids such as hydrochloride, hydrobromide, hydroiodide, sulphate, phosphate, diphosphate and nitrate, or with organic acids, such as acetate, maleate, fumarate, tartrate, succinate, citrate, lactate, methanesulphonate, p-toluenesulphonate, pamoate, oxalate and stearate. The salts formed from bases such as sodium or potassium hydroxide also fall within the scope of the present invention, when they can be used. For other examples of pharmaceutically acceptable salts, reference can be made to “Salt selection for basic drugs”, Int. J Pharm. (1986), 33, 201-217.

By simultaneous therapeutic use is meant in the present Application, an administration of several active ingredients by the same route and at the same time. By separate use is meant, in particular, an administration of several active ingredients at approximately the same time by different routes. By therapeutic administration over a period of time is meant the administration of several active ingredients at different times and in particular an administration method according to which the entire administration of one of the active ingredients is completed before the administration of the other or others begins. In this way it is possible to administer one of the active ingredients for several months before administering the other active ingredient or ingredients. In this case, no simultaneous administration occurs.

By ligands of opiate receptors or their salts is meant the substances chosen from naloxone, naltrexone, nalorphine, fentanyl, afentanil, codeine, dihydrocodeine, hydrocodone, oxycodone, hydromorphone, pethidine, remifentanyl, sufentanyl, dextropropoxyphene, tramadol, buprenorphine, nalbuphine, morphine, morphine sulphate, hydromorphone hydrochloride and coated morphine sulphate, as well as morphine derivatives.

By “sodium channel inhibitors”, is meant in particular the following compounds (optionally in the form of pharmaceutically acceptable salts):

carbamazepine;

lidocaine;

tetracaine;

bupivacaine;

procaine;

mepivacaine;

dibucaine;

lamotrigine;

mexiletine;

riluzole; or

butyl 2-(4-[1,1′-biphenyl]-4-yl-1H-imidazol-2-yl) ethylcarbamate (ICS compound).

By “non-steroidal anti-inflammatories (NSAID)”, is meant in particular the following compounds (optionally in the form of pharmaceutically acceptable salts):

acetylsalicylic acid and its derivatives;

indolic NSAIDs and derivatives such as for example indometacin and sulindac;

arylcarbocyclic NSAIDs such as tiaprofenic acid, alminoprofen, diclofenac, etodolac, flurbiprofen, ibuprofen, ketoprofen, nabumetone or naproxen;

NSAID oxicam derivatives such as meloxicam, piroxicam or tenoxicam;

morniflumate;

butazolidin;

selective inhibitors of cycloxygenase-2 (COX-2) such as celecoxib or rofecoxib, lumiracoxib, valdecoxib, deracoxib, parecoxib, etoricoxib and nimesulide; or

acetaminophen (paracetamol) and dipyrone.

By “inhibitors of the glutamatergic system” is meant in particular the following compounds (optionally in the form of pharmaceutically acceptable salts):

ketamine;

amantadine;

memantine; or

dextromethorphan.

By “tricyclic antidepressants” is meant in particular the following compounds (optionally in the form of pharmaceutically acceptable salts):

clomipramine;

amoxapine;

amitriptyline;

desipramine;

dothiepine hydrochloride;

doxepin;

imipramine;

nortriptyline;

protryptiline;

trimipramine

mirtazepine;

duloxetine; or

milnacipran.

By “alpha-2 adrenergic agonists” is meant in particular the following compounds (optionally in the form of pharmaceutically acceptable salts):

clonidine;

dexmedetomidine;

mivazerol;

lofexidine;

guanfacine; or

guanabenz acetate.

By “cannabinoids”, is meant in particular, without being limitative,

CP55940: ((−)-cis-3-[2-Hydroxy-4-(1,1-dimethylheptyl)phenyl]-trans-4(3-hydroxypropyl)cyclohexanol),

AM1241: (3-(2-Iodo-5-nitrobenzoly)-1-(1-methyl-2-piperidinylmethyl)-1H-indole,

WIN55212-2: (R)—(+)-[2,3-Dihydro-5-methyl-3[(morpholinyl)-methyl]pyrrolol[1,2,3-de]-1,4-benzoxazinyl]-(1-naphthalenyl)methadone mesylate or ((R)—(+)-[2,3-Dihydro-5-methyl-3-(4-morpholinylmethyl) pyrrolo[1,2,3-de]-1,4-benzoxazin-6-yl]-1-naphthalenylmethadone),

JWH-133: 3-(1′1′-Dimethylbityl)-1-deoxy-Δ⁸-tetrahydrocannabinol; 3-(1′,1′-Dimethykbutyl)-1-deoxy-Δ⁸-THC,

JWH-051: (2-Methyl-1-propyl-1H-indol-3-yl)-1-naphthalenylmethanone, or dronabinol.

By “GABAergic derivatives”, is meant in particular the following compounds (optionally in the form of pharmaceutically acceptable salts):

gabapentin;

baclofen; or

pregabalin.

By “pain” is meant in this Application “any unpleasant emotional and sensory experience associated with present or potential tissue damage or described in such terms by the patient ”.

A preferred variant of the invention relates to a product comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or one of its pharmaceutically acceptable salts in combination with morphine or a morphine analogue or derivative for a therapeutic use which is simultaneous, separate or spread out over time, for the treatment or prevention of pain.

In particular, the subject of the invention is a product comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or one of its pharmaceutically acceptable salts in combination with morphine for a therapeutic use which is simultaneous, separate or spread out over time for the treatment or prevention of pain. According to another variant of the invention (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or one of its pharmaceutically acceptable salts is combined with naloxone or naltrexone for a therapeutic use which is simultaneous, separate or spread out over time for the treatment or prevention of pain.

The invention also relates to a product comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or one of its pharmaceutically acceptable salts in combination with an analgesic agent chosen from sodium channel inhibitors, non-steroidal anti-inflammatories (NSAID), inhibitors of the glutamatergic system, tricyclic antidepressants and GABAergic derivatives for a therapeutic use which is simultaneous, separate or spread out over time for the treatment or prevention of pain.

Among the types of pain which can be treated by a product according to the invention, the following can be mentioned in particular:

pain associated with a cancer (particularly preferred inasmuch as (1R)-1-[({(2R)-2-amino-3-[(8S)-8-(cyclohexylmethyl)-2-phenyl-5,6-dihydroimidazo[1,2-α]pyrazin -7(8H)-yl]-3-oxopropyl}dithio)methyl]-2-[(8S)-8-(cyclohexylmethyl)-2-phenyl -5,6-dihydroimidazo[1,2-α]pyrazine-7(8H)-yl]-2-oxoethylamine is also an anti-cancer agent);

pain associated with chronic diseases other than a cancer such as pain associated with viral or retroviral diseases (for example pain associated with or following Acquired Immune Deficiency Syndrome (AIDS) or pain associated with shingles) or pain associated with diabetic neuropathies;

neuropathic pain such as trigeminal neuralgia, glosso-pharyngeal neuralgias, pain associated with radiculopathies, with diabetic neuropathies or anti-cancer agents, inflammatory pain and pain associated with secondary neuropathies with metastasic infiltrations;

adiposis dolorosa;

pain associated with bums;

migraine;

pre- and post-operative pain;

chronic inflammatory pain;

sciatica;

post-herpetic neuralgias;

chronic pain, fibromyalgia, algoneurodystrophy or complex regional pain syndrome.

central pain syndrome following vascular cerebral accidents, thalamic lesions or multiple sclerosis;

physical pain such as trauma, amputations;

or pain associated with intoxication.

Administration of a medicament according to the invention can be carried out by topical route, oral route, parenteral route, by intramuscular injection, sub-cutaneous injection, intravenous injection, etc. or by a combination of these routes.

The dose of a compound according to the present invention envisaged for the treatment of the diseases or disorders mentioned above, varies according to the administration method, the age and body weight of the subject to be treated as well as the subject's state, and it will be decided definitively by the attending doctor or veterinary surgeon. Such a quantity determined by the attending doctor or veterinary surgeon is here called “effective therapeutic quantity”.

Unless defined otherwise, all the technical and scientific terms used here have the same meaning as those commonly understood by an ordinary specialist in the field to which this invention belongs. Likewise, all the publications, patent applications, patents and other references mentioned here are incorporated by way of reference.

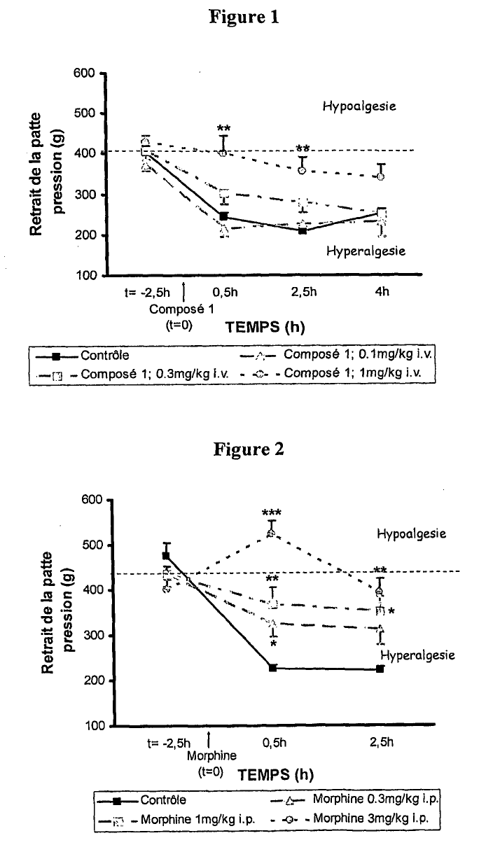

FIG. 1 shows the effect of compound (1) following injection by intravenous route in the carrageenin-induced hyperalgesia model.

FIG. 2 shows the effect of morphine following injection by intraperitoneal route in the carrageenin-induced hyperalgesia model.

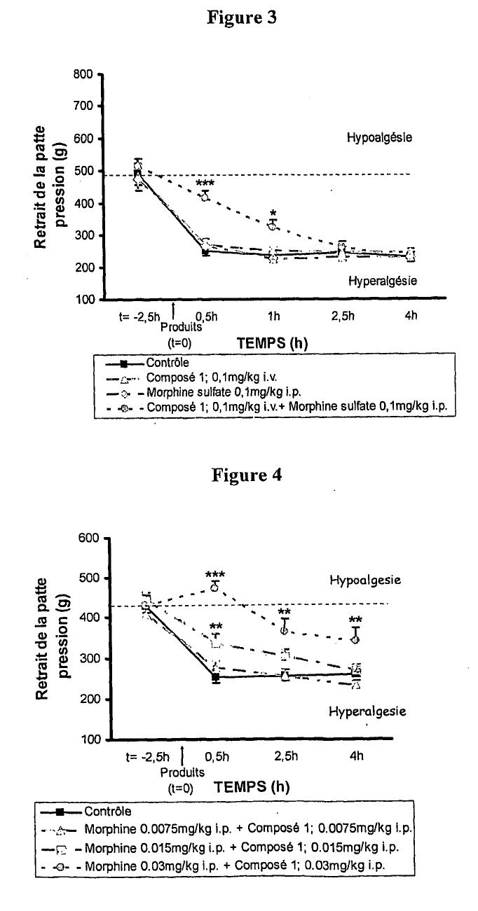

FIGS. 3 and 4 show the effect of the combination of compound (1) by intravenous route and morphine by intraperitoneal route in the carrageenin-induced hyperalgesia model.

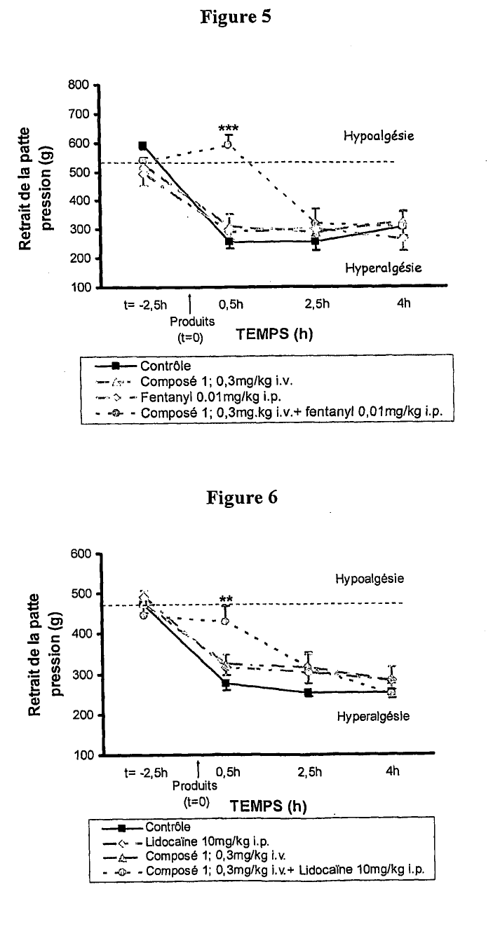

FIG. 5 shows the effect of fentanyl alone by intraperitoneal route and in combination with compound (1) by intravenous route in the carrageenin-induced hyperalgesia model.

FIG. 6 shows the effect of lidocaine alone by intraperitoneal route and in combination with compound (1) following injection by intravenous route in the carrageenin-induced hyperalgesia model.

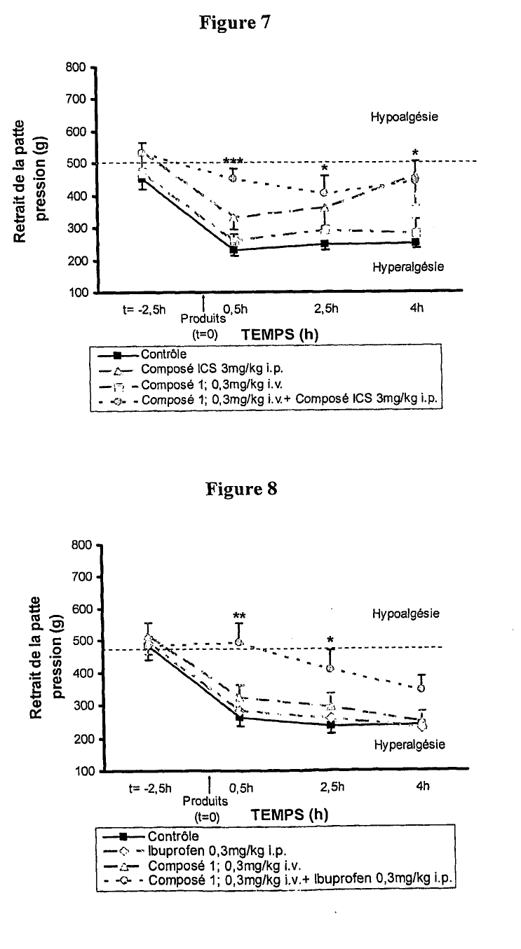

FIG. 7 shows the effect of a sodium channel inhibitor (ICS compound) by intraperitoneal route and in combination with compound (1) following injection by intravenous route in the carrageenin-induced hyperalgesia model.

FIG. 8 shows the effect of ibuprofen alone by intraperitoneal route and in combination with compound (1) following injection by intravenous route in the carrageenin-induced hyperalgesia model.

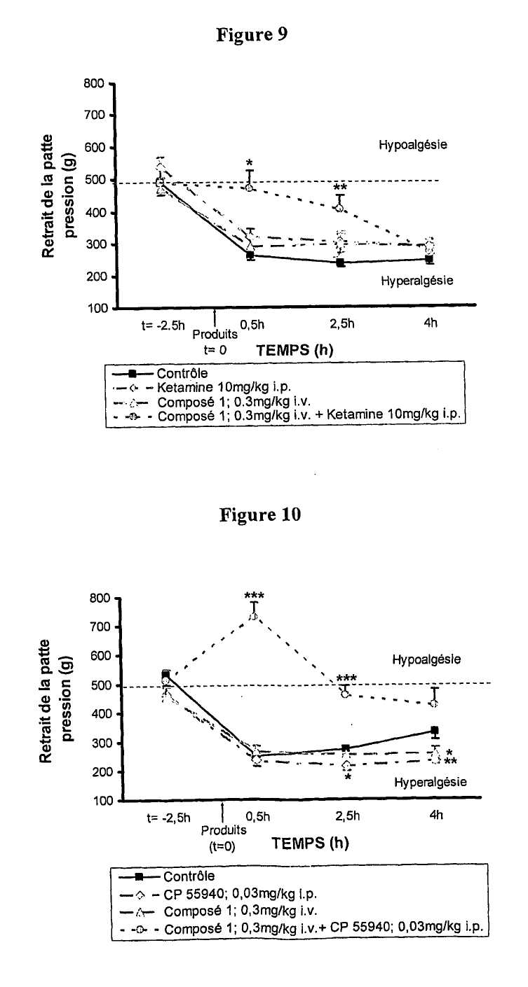

FIG. 9 shows the effect of ketamine alone by intraperitoneal route and in combination with compound (1) following injection by intravenous route in the carrageenin-induced hyperalgesia model.

FIG. 10 shows the effect of a cannabinoid receptor (CP 55940) by intraperitoneal route and in combination with compound (1) following injection by intravenous route in the carrageenin-induced hyperalgesia model.

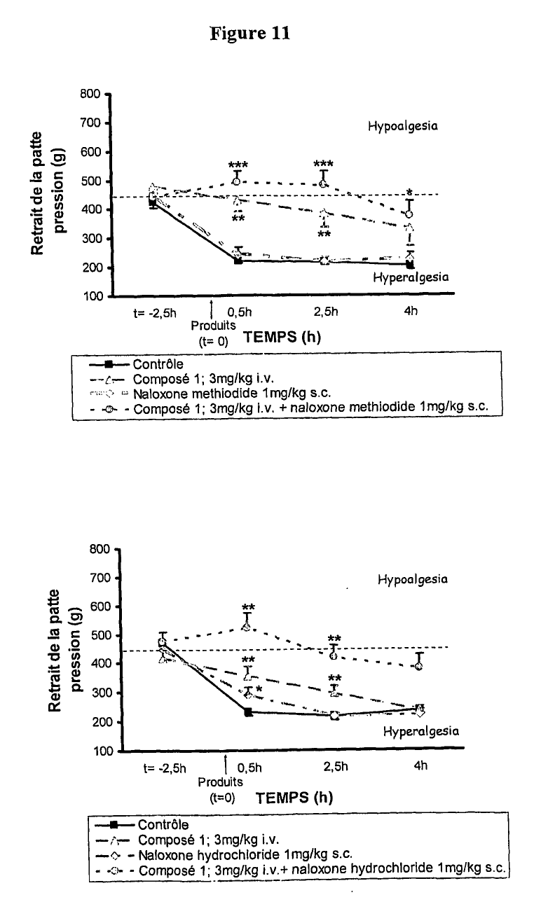

FIG. 11 shows the effect of a ligand of the opiate receptors alone (naloxone) by sub-cutaneous route and in combination with compound (1) following injection by intravenous route in the carrageenin-induced hyperalgesia model.

The following examples are given in order to illustrate the above procedures and must not under any circumstances be considered as limiting the scope of the invention.

Pharmacological Study of the Products of the Invention

The activity of the compounds of the invention was evaluated in vivo on a model of carrageenin-induced hyperalgesia on a rat's paw.

Male Sprague Dawley (Charles River) rats weighing 180 to 240 g on the day of the experiment are housed for 5 to 8 days under animal room conditions and fasted on grids for 18 hours before and during the experiment. The groups are constituted by at least 6 animals. The products are administered by intraperitoneal (i.p., 2 ml/kg) or intravenous route (i.v., 1 ml/kg), 2 hours 30 minutes after the injection of carrageenin. The 2% carrageenin was injected by sub-plantar route in the rear right paw of the rats. The nociceptive threshold was evaluated by measuring the withdrawal of the rat's paw caused by a mechanical stimulus applied using an analgesy meter (Randall-Selitto test). The measurements were taken just before the injection of carrageenin (t=−2 hours 30 minutes) and 30 minutes, 2 hours 30 minutes and 4 hours after the injection of the products to be tested (carried out at t=0). The effectiveness of the products is evaluated by their ability to reduce significantly the carrageenin-induced hyperalgesia. This effectiveness is statistically determined by a variance analysis test (one route) and/or a Dunnett's test (two routes).

In the examples hereafter, “compound 1” designates (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine (an inhibitor of the signal mediated by the heterotrimeric G proteins).

### Example 1

### Effect of Compound 1 on Carrageenin-Induced Hyperalgesia

The results obtained by using different doses of compound 1 in the model of carrageenin-induced hyperalgesia on a rat's paw as described above are shown in FIG. 1.

The analgesic activity of compound 1 is demonstrated in the test of carrageenin-induced hyperalgesia. Starting from a dose of 1 mg/kg (i.v.) the pain threshold following a mechanical stimulus applied to the rats' paws is significantly reduced.

### Example 2

### Effect of Morphine on Carrageenin-Induced Hyperalgesia

The results obtained by using different doses of morphine in the model of carrageenin-induced hyperalgesia on a rat's paw as described above are shown in FIG. 2.

The analgesic activity of the morphine is demonstrated in the carrageenin-induced hyperalgesia test. Starting from a dose of 1 mg/kg (i.p.) the pain threshold following a mechanical stimulus applied to the rats' paws is significantly reduced.

### Example 3

### Effect of the Combination of Compound 1 and Morphine on Carrageenin-Induced Hyperalgesia

The results obtained by using different combinations of compound 1 and morphine in the model of carrageenin-induced hyperalgesia on a rat's paw described above are shown in FIGS. 3 and 4.

The analgesic activity of the combination of compound 1+ morphine is demonstrated in the same model as previously. The analgesic effect of this combination is more powerful and more durable than the effects of each of the compounds used alone (in other words, a synergic effect is observed). A dose of 0.015 mg/kg (i.p.) morphine in the context of such a combination is found to be effective in this case.

In comparison with the results of Examples 1 and 2, it is noted therefore that the administration of compound 1 and morphine has a synergic analgesic effect when in combination (FIG. 4). In fact, the use of compound 1 allows the doses of morphine necessary in order to obtain an equivalent effect to be reduced by at least a factor of 30-50.

### Example 4

### Effect of the Combination of Compound 1 and Fentanyl on Carrageenin-Induced Hyperalgesia

The results obtained by using different combinations of compound 1 and fentanyl in the model of carrageenin-induced hyperalgesia on a rat's paw described above are shown in FIG. 5.

A synergic effect is noted when fentanyl is combined with compound (1). In fact at time 0.5 hours, the combination allows the pain threshold tolerated by the rat on its inflamed paw when increasing pressure in applied to be increased. The pain threshold which was approximately 250-300 g/mm² for the control or compound (1) alone, is 600 g/mm² under the effect of the combination.

### Example 5

### Effect of the Combination of Compound 1 and Lidocaine on Carrageenin-Induced Hyperalgesia

The results obtained by using different combinations of compound 1 and lidocaine in the model of carrageenin-induced hyperalgesia on the rat's paw described above are shown in FIG. 6.

A synergic effect is observed when lidocaine is combined with compound (1). In fact at time 0.5 hours, the combination allows the pain threshold tolerated by the rat on its inflamed paw when increasing pressure is applied to be increased. The pain threshold which was approximately 280-320 g/mm² for the control or compound (1) alone, is 430 g/mm² under the effect of the combination.

### Example 6

### Effect of the Combination of Compound 1 and a Sodium Channel Inhibitor (ICS compound) on Carrageenin-Induced Hyperalgesia

The results obtained by using different combinations of compound 1 and a sodium channel inhibitor in the model of carrageenin-induced hyperalgesia on a rat's paw as described above are shown in FIG. 7.

The sodium channel inhibitor used is butyl 2-[4-(1,1′-biphenyl-4-yl)-1H-imidazol -2-yl]ethylcarbamate (called ICS compound).

A synergic effect is observed when butyl 2-[4-(1,1′-biphenyl-4-yl)-1H-imidazol -2-yl]ethylcarbamate is combined with compound (1). In fact at time 0.5 hours, the combination allows the pain threshold tolerated by the rat on its inflamed paw when increasing pressure is applied to be increased. The pain threshold which was approximately 250 g/mm² for the control or compound (1) alone, is 450 g/mm² under the effect of the combination.

### Example 7

### Effect of the Combination of Compound 1 and Ibuprofen on Carrageenin-Induced Hyperalgesia

The results obtained by using different combinations of compound 1 and ibuprofen in the model of carrageenin-induced hyperalgesia on a rat's paw described above are shown in FIG. 8.

A synergic effect is observed when ibuprofen is combined with compound (1). In fact at time 0.5 hours, the combination allows the pain threshold tolerated by the rat on its inflamed paw when increasing pressure is applied to be increased. The pain threshold which was approximately 260-320 g/mm² for the control or compound (1) alone, is 500 g/mm² under the effect of the combination.

### Example 8

### Effect of the Combination of Compound 1 and Ketamine on Carrageenin-Induced Hyperalgesia

The results obtained by using different combinations of compound 1 and ketamine in the model of carrageenin-induced hyperalgesia on the rat's paw described above are shown in FIG. 9.

A synergic effect is observed when ketamine is combined with compound (1). In fact at time 0.5 hours, the combination allows the pain threshold tolerated by the rat on its inflamed paw when increasing pressure is applied to be increased. The pain threshold which was approximately 260-290 g/mm² for the control or compound (1) alone, is 480 g/mm² under the effect of the combination.

### Example 9

### Effect of the Combination of Compound 1 and a Cannabinoid Receptor Agonist (CP55940) on Ccarrageenin-Induced Hyperalgesia

The results obtained by using different combinations of compound 1 and a cannabinoid receptor agonist in the model of carrageenin-induced hyperalgesia on the rat's paw described above are shown in FIG. 10.

A synergic effect is observed when CP 55940 is combined with compound (1). In fact at time 0.5 hours, the combination allows the pain threshold tolerated by the rat on its inflamed paw when increasing pressure is applied to be increased. The pain threshold which was approximately 260 g/mm² for the control or compound (1) alone, is 730 g/mm² under the effect of the combination.

### Example 10

### Effect of the Combination of Compound 1 and a Ligand of Opiate Receptors on Carrageenin-Induced Hyperalgesia

The results obtained by using different combinations of compound 1 and a ligand of opiate receptors in the model of carrageenin-induced hyperalgesia on the rat's paw described above are shown in FIG. 11.

The ligand of opiate receptors used is naloxone.

A potentialization is observed when naloxone is combined with compound (1). In fact at time 0.5 hours, the combination allows the pain threshold tolerated by the rat on its inflamed paw when increasing pressure is applied to be increased. The pain threshold which was approximately 220 g/mm² for the control and 430 for compound (1) alone (3 mg/kg i.v.), is 500 g/mm² under the effect of the combination.

1. Product comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or a pharmaceutically acceptable salt thereof and an analgesic agent including ligands of opiate receptors or salts thereof.

2. Product according to claim 1, comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or a pharmaceutically acceptable salt thereof and morphine or an analogues or derivatives thereof.

3. Product according to claim 1, wherein the ligands of opiate receptors or their salts are naloxone, naltrexone, nalorphine, fentanyl, alfentanil, codeine, dihydrocodeine, hydrocodone, oxycodone, hydromorphone, pethidine, remifentanyl, sufentanyl, dextropropoxyphene, tramadol, buprenorphine, nalbuphine, morphine, morphine sulphate, hydromorphone hydrochloride, coated morphine sulphate, or morphine derivatives.

4. Product according to claim 1, comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio)methyl]-2-[(8S)-8-(cyclohexylmethyl)-2-phenyl -5,6-dihydroimidazo [1,2-α]pyrazin-7(8H)-yl]-2-oxoethylamine or a pharmaceutically acceptable salt thereof and naloxone or naltrexone.

5. Product according to claim 2, consisting of (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or a pharmaceutically acceptable salt thereof and morphine.

6. The method according to claim 15, comprising administering the combination simultaneously.

7. The method according to claim 15, comprising administering the combination separately.

8. The method according to claim 15, comprising administering the combination spread out over time.

9. The method according to 14, wherein the pain treated is pain associated with a cancer, pain associated with chronic diseases other than a cancer, neuropathic pain, pain associated with radiculopathies, with diabetic neuropathies or associated with AIDS or following AIDS, or anticancer agents, inflammatory pain, adiposis dolorosa, pain associated with burns, migraine, pre-operative pain, post-operative pain, chronic inflammatory pain, sciatica, post-herpetic neuralgias, fibromyalgia, algoneurodystrophy or complex regional pain syndrome, central pain following cerebral vascular accidents, thalamic lesions or multiple sclerosis or physical pain or pain associated with intoxication.

10. The method according to claim 9, wherein the pain treated is associated with a cancer.

11. The method according to claim 9, wherein the pain treated is associated with a chronic disease other than a cancer.

12. Product according to claim 1, wherein the ligands of opiate receptors or salts thereof are morphine or morphine derivatives, sodium channel inhibitors, non-steroidal anti-inflammatories (NSAID), glutamatergic system inhibitors, tricyclic antidepressants or GABAergic derivatives.

13. The method according to claim 9, wherein the physical pain is pain associated with trauma or amputations.

14. A method for prevention or treating of pain comprising administering a product comprising (1R)-1-[({(2R)-2-amino-3-[(8S)-8-(cyclohexylmethyl)-2-phenyl-5,6-dihydroimidazo [1,2-α]pyrazin-7(8H)-yl]-3-oxopropyl}dithio) methyl]-2-[(8S)-8-(cyclohexylmethyl)-2-phenyl-5,6-dihydroimidazo [1,2-α]pyrazin-7(8H)-yl]-2-oxoethylamine or a pharmaceutically acceptable salt thereof in combination with an analgesic agent including ligands of opiate receptors or salts thereof.

15. The method of claim 14, comprising administering the combination simultaneously, separate or spread out over time.
